(19) United States
(12) Patent Application Publication
(10) Pub. No.: US 2008/0125652 A1
(43) Pub. Date: May 29, 2008

(54) Method and System for Deriving a Fetal Heart Rate Without the Use of an Electrocardiogram in Non-3d Imaging Applications

(75) Inventor: David W. Clark, Windham, NH (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N.V., Eindhoven (NL)

(21) Appl. No.: 11/814,854

(22) PCT Filed: Jan. 26, 2006
(86) PCT No.: PCT/IB06/50293

§ 371 Date: Jul. 26, 2007

Related U.S. Application Data
(60) Provisional application No. 60/648,484, filed on Jan. 31, 2005.

(51) Int. Cl. A61B 8/02

(52) U.S. Cl. 600/437

(57) ABSTRACT

A system and method are provided for determining a fetal heart rate from ultrasound imaging data in near real-time. The heart rate is determined by analyzing spatial points on ultrasound cardiac volumes and calculating the peak spectral frequency of the changes in ultrasonic characteristics of the spatial points.

The present invention relates generally to medical devices. More particularly, the present invention relates to a method and device for deriving a heart rate without the use of an electrocardiogram in non-3D imaging applications.

Medical ultrasound imaging has become a popular means for visualizing and medically diagnosing the condition and health of interior regions of the human body. With this technique, an acoustic transducer probe, which is attached to an ultrasound system console via an interconnection cable, is held against the patient's tissue by the sonographer where it emits and receives focused ultrasound waves in a scanning fashion. The scanned ultrasound waves, or ultrasound beams, allow the systematic creation of image slices of the patient's internal tissues for display on the ultrasound console. The technique is generally quick, painless, fairly inexpensive and safe, even for such uses as fetal imaging.

Ultrasound imaging systems commonly in use generate and transmit ultrasound signals to map internal tissue typography, vascular fluid flow rates, and abnormalities. The systems typically incorporate several methods, or modes, of imaging, i.e. Brightness Mode (B-Mode), Harmonic, Spectral Doppler, and Color Flow.

Each imaging method has its characteristic uses and limitations. B-Mode imaging is typically used to image the structure of internal tissue and organs with high spatial resolution. Generally to achieve this degree of spatial resolution, short-duration ultrasound pulses are advantageous. Harmonic imaging uses the harmonic frequencies produced from nonlinear wave propagation. Harmonic imaging can reduce clutter, sidelobes, and aberration compared to the more traditional fundamental B-mode imaging, but typically involves compromising spatial resolution.

Color Flow imaging is primarily used to image blood flow and locate abnormal or turbulent flows within the cardiovascular system. Color Flow images are usually overlaid on to a B-Mode structural image. However, the ultrasound properties necessary for proper Color Flow imaging differ from those used in B-Mode. Color Flow imaging requires multiple pulses to detect motion, and longer duration ultrasound pulses than commonly used for B-Mode scans for sensitivity. Low ultrasound pulse repetition rates are desirable for slow-flowing veins, but for the faster flows found in the arteries and heart, higher ultrasound pulse repetition rates are necessary to properly avoid aliasing errors.

Spectral Doppler uses a very large number of ultrasound pulses (or a continuous wave) in the same direction, and converts the resulting echo data stream into a frequency spectrum versus time display and an audio output. Spectral Doppler provides more detailed blood flow dynamics information for one location, in contrast to Color Flow's simple estimation for many locations. Typically Color Flow is used to decide where to place the Spectral Doppler sample location.

3D ultrasound imaging involves scanning the ultrasound pulses over a volume rather than one plane, either mechanically or electrically. Typically the volume is scanned as a series of 2D planes. The echo data is usually displayed either as a volume-rendered image or as 2D planar images sliced through the volume data. The name 4D imaging is sometimes used when 3D volumes are acquired and/or displayed rapidly enough to see motion of the structure being imaged (time being the fourth dimension).

The heart and the fetus have been the two main applications for 3D ultrasound imaging, because both involve significant volumes of liquid that are nearly transparent to ultrasound, so the anatomy can be visualized relatively easily in three dimensions. Particularly with color flow imaging, a 3D acquisition is typically too slow for the fast motion of the heart, so for adult or pediatric 3D cardiac exams an electrocardiogram is used to synchronize the ultrasound acquisition over multiple cardiac cycles. An electrocardiogram is impractical for a fetal heart exam, however. Nelson, Sklansky, and Pretorius at the University of California at San Diego published a technique for deriving the fetal heart rate from the 2D B-mode images in a slow (many heart cycle) 3D scan through the fetal heart, then using the derived heart rate to shuffle the 2D images into 3D volumes of the fetal heart at multiple points of the cardiac cycle. That technique (called the NSP technique hereafter) is implemented in several commercially available ultrasound systems, and will be described in more detail below. The NSP technique has only been applied to post-processing 3D image acquisitions of the fetal heart, and it depends on having significant cardiac motion in the 2D B-mode images.

An objective of the present disclosure is to extend the NSP technique to derive, display or use the heart rate without needing an electrocardiogram, in situations other than 3D imaging of the fetal heart. A further objective of the present disclosure is to extend the NSP technique to operate on data other than 2D B-mode slices of a 3D acquisition, which is particularly useful in non-cardiac exams where there is very little cardiac-cycle motion in the B-mode images and electrocardiograms are seldom used. A further objective of the present disclosure is to extend the NSP technique to operate repetitively, including using overlapping time segments, to provide rapidly updated heart rate estimates in a live imaging situation.

An embodiment of the present disclosure provides a method for determining a heart rate from a set of ultrasound images, in situations other than 3D imaging of a fetal heart. The estimated heart rate is the frequency of the peak of the summed power spectra of a subset of spatial points over a set of ultrasound images. The derived heart rate can be used to set the time span of a repetitive loop display to an integral number of heartbeats, or to combine data from multiple heartbeats for noise reduction or temporal resolution improvement, or to display the heart rate numerically in a live imaging situation, or to reconstruct a 3D or 4D volume in applications other than fetal heart. The heart rate can be derived from ultrasound data other than fetal cardiac elevation-swept B-mode images, such as M-mode, cardiac or arterial color flow correlations or velocities, or spectral Doppler data.

An additional embodiment of the present disclosure provides for an ultrasound medical imaging system. The ultrasound medical imaging system includes an ultrasound imager having an ultrasound transducer, a processor, and a video display.

These and other features, aspects, and advantages of the present invention will become better understood with regard to the following description, appended claims, and accompanying drawings wherein:

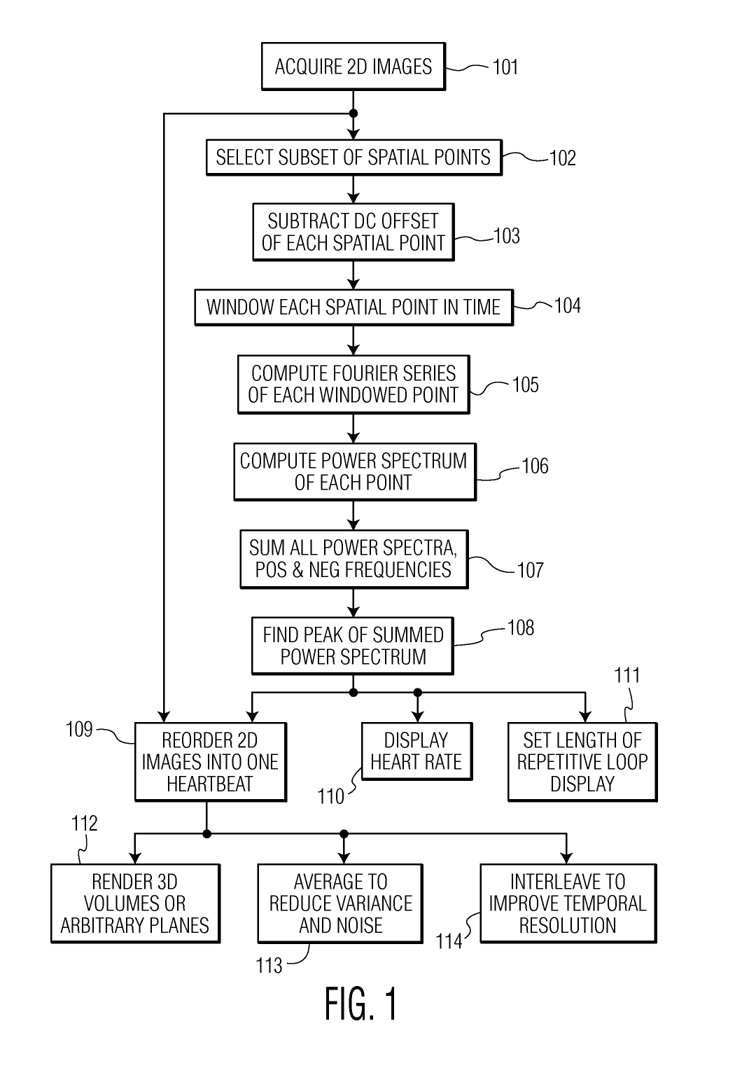

FIG. 1 is a flow chart illustrating the steps for determining a heart rate from a set of ultrasound images in accordance with an embodiment of the present disclosure;

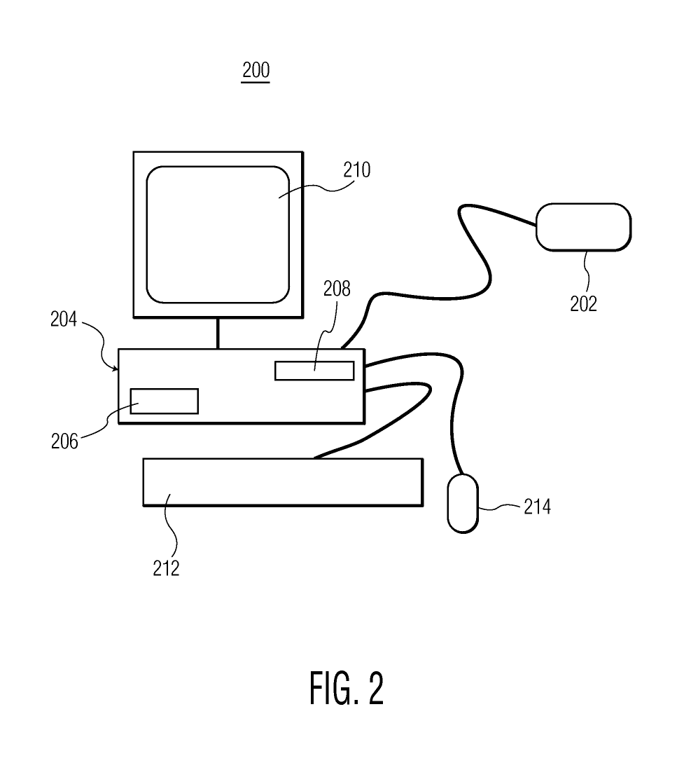

FIG. 2 is a schematic view illustrating an ultrasound imaging system in accordance with an embodiment of the present disclosure; and

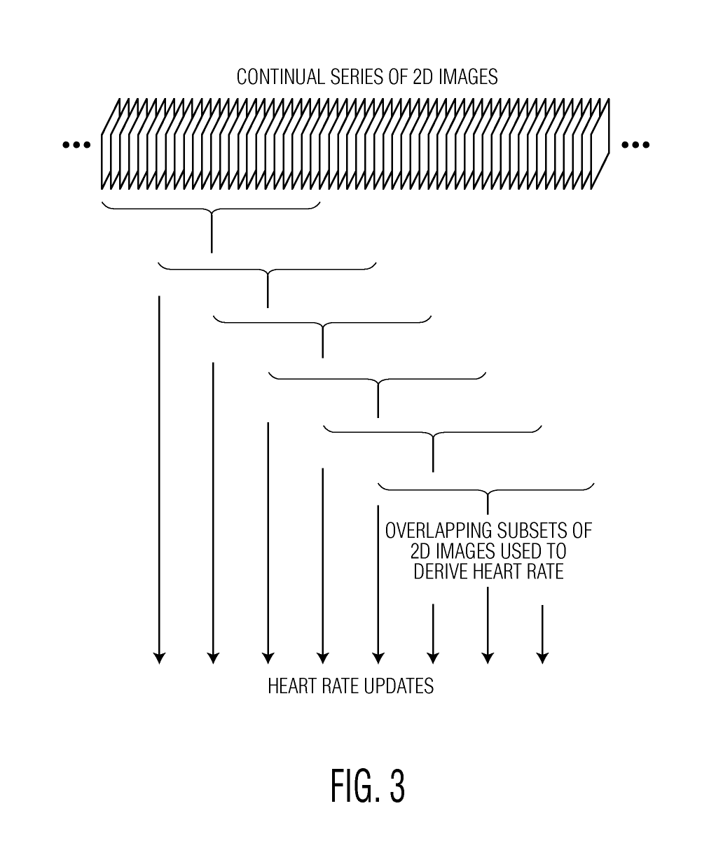

FIG. 3 is a diagram illustrating the overlapping derivation of heart rate in a continual imaging situation.

As shown in FIG. 1, an embodiment of the present disclosure provides a method for determining a heart rate from a set of ultrasound cardiac images. In step 101, an ultrasound imaging device begins a scan to acquire 2D cardiac images (later it will be described how this can be other than 2D cardiac images). Proceeding on, step 102 selects a subset of spatial points, such as a uniformly spaced grid. The selection step may be performed either manually or as an automated process. In step 103, the DC offset and slow variation is removed from each spatial point. Proceeding to step 104, the spatial points selected in step 102 are plotted with respect to time and a window function is applied to the data in step 105. Two appropriate window functions are the Hann and Hamming functions. A power spectrum is calculated in step 106 for the windowed data. In step 107, all the power spectra are summed, including both positive and negative frequencies. From the summed power spectra, a power spectrum peak is derived and processed along with the time sampling rate between image scans to determine the heart rate in step 108. The summed power spectrum covers the frequency range from zero to half the sample rate (the sample rate is the 2D frame rate). The location of the peak of the power spectrum is therefore at some fraction of the sample rate. Multiplying that by the sample rate in Hertz gives the heart rate in Hertz, and multiplying by 60 gives the heart rate in beats per minute.

There are several alternative ways that the derived heart rate may be used, as shown in FIG. 1. The prior art uses the heart rate to rearrange slowly elevation-swept 2D images of a fetal heart into multiple 3D volumes at different times of the heart cycle, step 109.

Other alternative uses of the derived heart rate, shown in FIG. 1, are a subject of the present disclosure. If the 2D images are elevation-swept, they may be rearranged into multiple 3D volumes in applications other than fetal heart. The length of a repetitive loop display of some subset of the 2D images (typically called a cine-loop) can be set to a whole number of cardiac cycles to minimize discontinuities when the loop wraps from end back to beginning, step 111. The reordered images from step 109 can be averaged to reduce variance and noise, step 113. The reordered images from step 109 can also be interleaved to improve temporal resolution, step 114.

The derived heart rate may also be displayed numerically, which is particularly useful when it is done repetitively in a live imaging situation. To provide a more rapidly updating heart rate display while still using several heartbeats of data to derive the heart rate accurately, the time spans of data for each heart rate estimate may be overlapped, as shown in FIG. 3. For example, a 4 second time span can be stepped along every 0.5 second.

A further subject of the present disclosure is to use data other than fetal heart swept-elevation B-mode images to derive the heart rate. The data could be B-mode images of a non-fetal heart, or B-mode images of a fetal heart without elevation sweeping. The data could also be M-mode data, either grayscale or color. The data could also be color flow data of a heart or artery, either complex correlations or detected velocities. The data could also be Doppler spectra.

Another embodiment of the present disclosure, shown in FIG. 2, provides for a medical ultrasound imaging system 200. The system 200 includes an ultrasound transducer assembly 202 connected to an imaging workstation 204. The imaging workstation 204 contains one or more processors 206 and at least one storage device 208, such as a hard drive, RAM disk, etc. The storage device(s) 208 may be used for storing the controlling and imaging software for the ultrasound system 200 as well as providing temporary and long term storage of image data acquired by the ultrasound transducer 202. The ultrasound imaging system 200 also provides a video display 210 and user input devices, including a keyboard 212 and a mouse 214.

The processor 206 is configured to execute the controlling and imaging software. The imaging software allows the operator of the system 200 to visualize and manipulate the data received from the ultrasound transducer 202. Additionally, the imaging software includes subroutines to perform the method of the present disclosure as exemplified by FIG. 1 and described in detail above.

In short, the processor 206 executes a set of programmable instructions for performing the method of the present disclosure. It is contemplated that the set of programmable instructions can be stored within the at least one storage device 208 and/or on a computer-readable medium, such as a CD, 3.5″ diskette, hard drive, etc., and capable of being executed by the processor 206.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. A method for determining a heart rate from a set of ultrasound images comprising the steps of:
acquiring a set of ultrasound images;
selecting a subset of spatial points from said set of ultrasound images;
processing data corresponding to each of said selected spatial points, including computing a power spectrum for each of said selected spatial points and summing all power spectrum; and
determining a spectral peak frequency for said summed power spectra, wherein said determined spectral peak frequency is substantially identical to said heart rate.

2. The method according to claim 1, further comprising the step of subtracting a DC offset from each of said selected spatial points.

3. The method according to claim 1, further comprising the steps of:
plotting each of said selected spatial points with respect to time; and
applying a window function to said data.

4. The method according to claim 3, wherein the step of processing data comprises the step of computing said power spectrum for said data windowed by said applying a window step.

5. The method according to claim 1, further comprising the step of displaying said heart rate.

6. A system for determining a heart rate from a set of ultrasound images comprising:
means for acquiring a set of ultrasound images;
means for selecting a subset of spatial points from said set of ultrasound images;
means for processing data corresponding to each of said selected spatial points, including means for computing a power spectrum for each of said selected spatial points and means for summing all power spectrum; and
means for determining a spectral peak frequency for said summed power spectra, wherein said determined spectral peak frequency is substantially identical to said heart rate.

7. The system according to claim 6, further comprising means for subtracting a DC offset from each of said selected spatial points.

8. The system according to claim 6, further comprising the steps of:
means for plotting each of said selected spatial points with respect to time; and
means for applying a window function to said data.

9. The system according to claim 8, wherein the means for computing said power spectrum uses said data windowed by said means for applying a window.

10. The system according to claim 6, further comprising means for displaying said heart rate.

11. A computer readable medium storing a set of programmable instructions for being executed by a processor (206) for performing a method for determining heart rate from a set of ultrasound images comprising the steps of:
acquiring a set of ultrasound images;
selecting a subset of spatial points from said set of ultrasound images;
processing data corresponding to each of said selected spatial points, including computing a power spectrum for each of said selected spatial points and summing all power spectrum; and
determining a spectral peak frequency for said summed power spectra, wherein said determined spectral peak frequency is substantially identical to said heart rate.

12. The computer readable medium according to claim 11, wherein the method further comprises the step of subtracting a DC offset from each of said selected spatial points.

13. The computer readable medium according to claim 11, wherein the method further comprises the steps of:
plotting each of said selected spatial points with respect to time; and
applying a window function to said data.

14. The computer readable medium according to claim 13, wherein the step of processing data comprises the step of computing said power spectrum for said data windowed by said applying a window step.

15. The computer readable medium according to claim 11, wherein the method further comprises the step of displaying said heart rate.
